(19) United States
(12) Patent Application Publication
(10) Pub. No.: US 2023/0240767 A1
(43) Pub. Date: Aug. 3, 2023

(54) TECHNIQUES FOR SELECTIVE JOINT FLOATING IN A COMPUTER-ASSISTED SYSTEM

(71) Applicant: INTUITIVE SURGICAL OPERATIONS, INC., Sunnyvale, CA (US)

(72) Inventor: Daniel H. GOMEZ, Los Gatos, CA (US)

(21) Appl. No.: 18/010,798

(22) PCT Filed: Jun. 15, 2021
(86) PCT No.: PCT/US2021/037506

§ 371 Date: Dec. 15, 2022

Related U.S. Application Data
(60) Provisional application No. 63/039,871, filed on Jun. 16, 2020.

(51) Int. Cl. A61B 34/30; A61B 90/00

(52) U.S. Cl. CPC A61B 34/30; A61B 90/06; A61B 2034/306; A61B 2090/064

(57) ABSTRACT

Techniques of selective joint floating in a computer-assisted system include a computer-assisted device that includes a kinematic chain including a plurality of links coupled by a plurality of joints and a control unit coupled to the kinematic chain. The kinematic chain is configured to support an end effector. The control unit is configured to determine location information for a first operator interaction with the kinematic chain, determine one or more joints to place into a floating state based on one or more parameters, and place the one or more joints into the floating state in response to determining the one or more joints. The one or more parameters being of the first operator interaction or of computer-assisted device. The one or more parameters including the location information. The plurality of joints including the one or more joints.

### CROSS-REFERENCES TO RELATED APPLICATIONS

The present claims priority to U.S. Provisional Patent Application No. 63/039,871 filed Jun. 16, 2020 and titled “Techniques for Selective Joint Floating in a Computer-assisted System.” The disclosure of which is incorporated herein by reference in its entirety.

### TECHNICAL FIELD

The present disclosure relates generally to operation of devices with repositionable arms and more particularly to a system and method for selective joint floating in a computer-assisted system.

### BACKGROUND

Computer-assisted devices with repositionable elements often include a kinematic chain, such as a kinematic chain comprising a repositionable arm configured to support a distally mounted instrument or end effector. In many situations, the position and orientation of the distal end effector is controlled to perform a task, such as while the distal end effector is inserted into a patient, in a medical example. Generally, the kinematic chain includes one or more actuated and/or non-actuated joints coupling one or more links, and movement of the one or more joints modifies the configuration of the kinematic chain, and can modify the positioning and orientation of the distal end effector relative to a proximal base of the kinematic chain. In practice, during the setup of the computer-assisted device for use to perform a task, such as part of a surgical procedure, the kinematic chain may be configured to place the distal end effector where it will perform the task.

To facilitate reconfiguration of a kinematic chain, some computer-assisted devices may place at least one joint into a floating (or “clutched”) state, in which one or more of the brakes and/or actuators for the at least one joint of the kinematic chain are released and/or commanded to facilitate external articulation of the at least one joint. The floating state enables an operator to manually change the positions and/or the orientations of certain portions of the kinematic chain via direct manipulation.

Accordingly, improved methods and systems for facilitating external articulation of joints in computer-assisted devices are desirable.

### SUMMARY

Consistent with some embodiments, a computer-assisted device that includes a kinematic chain including a plurality of links coupled by a plurality of joints and a control unit coupled to the kinematic chain. The kinematic chain is configured to support an end effector. The control unit is configured to determine location information for a first operator interaction with the kinematic chain, determine one or more joints to place into a floating state based on one or more parameters, and place the one or more joints into the floating state in response to determining the one or more joints. The one or more parameters being of the first operator interaction or of computer-assisted device. The one or more parameters including the location information. The plurality of joints including the one or more joints.

Consistent with some embodiments, a method performed by a control unit of a computer-assisted device includes determining location information for a first operator interaction with a kinematic chain of the computer-assisted device. The kinematic chain includes a plurality of links coupled by a plurality of joints. The kinematic chain is configured to support an end effector. The method further includes determining one or more joints to place into a floating state based on one or more parameters and placing the one or more joints into the floating state in response to determining the one or more joints. The one or more parameters being of the first operator interaction or of computer-assisted device. The one or more parameters including the location information. The plurality of joints including the one or more joints.

Consistent with some embodiments, a non-transitory machine-readable medium including a plurality of machine-readable instructions which when executed by one or more processors are adapted to cause the one or more processors to perform any of the methods described herein.

### BRIEF DESCRIPTION OF THE DRAWINGS

FIG. 1 is a simplified diagram of a computer-assisted system according to some embodiments.

FIGS. 2A-2D are simplified schematic views that illustrate various computer-assisted device system architectures for a computer-assisted device with one or more repositionable arms, according to various embodiments.

FIG. 3 is a simplified diagram showing a kinematic chain comprising a repositionable arm according to some embodiments.

FIG. 4 is a schematic diagram of a kinematic chain configured according to some embodiments.

FIG. 5 is a simplified diagram of a method of placing joints of a computer-assisted system into a floating state, according to some embodiments.

FIG. 6 schematically illustrates the kinematic chain of FIG. 4 responding to an operator interaction with a single contact location, according to some embodiments.

FIG. 7 schematically illustrates the kinematic chain of FIG. 4 responding to an operator interaction with a pushing action at a single contact location when no constraint is present, according to some embodiments.

FIG. 8 schematically illustrates the kinematic chain of FIG. 4 responding to an operator interaction with two contact locations when no constraint is present, according to some embodiments.

FIG. 9 schematically illustrates the kinematic chain of FIG. 4 responding to an operator interaction with two contact locations when a constraint is present, according to some embodiments.

FIG. 10 schematically illustrates the kinematic chain of FIG. 4 responding to an operator interaction with two contact locations when a constraint is present, according to some embodiments.

In the figures, elements having the same designations have the same or similar functions.

### DETAILED DESCRIPTION

In this description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional.

Further, this description's terminology is not intended to limit the invention. For example, spatially relative terms-such as “beneath”, “below”, “lower”, “above”, “upper”, “proximal”, “distal”, and the like-may be used to describe one element's or feature's relationship to another element or feature as illustrated in the figures. These spatially relative terms are intended to encompass different positions (i.e., locations) and orientations (i.e., rotational placements) of the elements or their operation in addition to the position and orientation shown in the figures. For example, if the content of one of the figures is turned over, elements described as “below” or “beneath” other elements or features would then be “above” or “over” the other elements or features. Thus, the term “below,” for example, can encompass both positions and orientations of above and below. A device may be otherwise oriented (rotated 90 degrees or at other orientations) and the spatially relative descriptors used herein interpreted accordingly. Likewise, descriptions of movement along and around various axes include various special element positions and orientations. In addition, the singular forms “a”, “an”, and “the” are intended to include the plural forms as well, unless the context indicates otherwise. And, the terms “comprises”, “comprising”, “includes”, and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components.

Elements described in detail with reference to one embodiment, implementation, or module may, whenever practical, be included in other embodiments, implementations, or modules in which they are not specifically shown or described. For example, if an element is described in detail with reference to one embodiment and is not described with reference to a second embodiment, the element may nevertheless be claimed as included in the second embodiment. Thus, to avoid unnecessary repetition in the following description, one or more elements shown and described in association with one embodiment, implementation, or application may be incorporated into other embodiments, implementations, or aspects unless specifically described otherwise, unless the one or more elements would make an embodiment or implementation non-functional, or unless two or more of the elements provide conflicting functions.

In some instances, well known methods, procedures, components, and circuits have not been described in detail so as not to unnecessarily obscure aspects of the embodiments.

This disclosure describes various devices, elements, and portions of computer-assisted devices and elements in terms of their state in three-dimensional space. As used herein, the term “position” refers to the location of an element or a portion of an element in a three-dimensional space (e.g., three degrees of translational freedom along Cartesian x-, y-, and z-coordinates). As used herein, the term “orientation” refers to the rotational placement of an element or a portion of an element (three degrees of rotational freedom—e.g., roll, pitch, and yaw, angle-axis, rotation matrix, quaternion representation, and/or the like). As used herein, the term “shape” refers to a set of positions and/or orientations measured along an element. As used herein, and for a computer-assisted device with repositionable arms, the term “proximal” refers to a direction toward the base of the computer-assisted device along its kinematic chain and “distal” refers to a direction away from the base along the kinematic chain. As used herein, the term “pose” refers to the six degree of freedom (DOF) spatial position and orientation of an element or a portion of an element.

Aspects of this disclosure are described in reference to computer-assisted systems and devices, which may include systems and devices that are teleoperated, remote-controlled, autonomous, semiautonomous, robotic, and/or the like. Further, aspects of this disclosure are described in terms of an implementation using a surgical system, such as the da Vinci® Surgical System commercialized by Intuitive Surgical, Inc. of Sunnyvale, California. Knowledgeable persons will understand, however, that inventive aspects disclosed herein may be embodied and implemented in various ways, including robotic and, if applicable, non-robotic embodiments and implementations. Implementations on da Vinci® Surgical Systems are merely examples and are not to be considered as limiting the scope of the inventive aspects disclosed herein. In some embodiments, the instruments, systems, and methods described herein may be suitable for use in, for example, surgical, teleoperated surgical, diagnostic, therapeutic, or biopsy procedures. While some embodiments are provided herein with respect to such procedures, any reference to medical or surgical instruments and medical or surgical methods is intended as non-limiting. Thus, the instruments, systems, and methods described herein may be used for humans, animals, portions of human or animal anatomy, non-surgical diagnosis, as well as for industrial systems, general robotic, or teleoperational systems. As further examples, the instruments, systems, and methods described herein may be used for non-medical purposes including industrial uses, general robotic uses, sensing or manipulating non-tissue work pieces, cosmetic improvements, imaging of human or animal anatomy, gathering data from human or animal anatomy, setting up or taking down systems, training medical or non-medical personnel, and/or the like. Additional example applications include use for procedures on tissue removed from human or animal anatomies (without return to a human or animal anatomy) and for procedures on human or animal cadavers. Further, these techniques can also be used for medical treatment or diagnosis procedures that include, or do not include, surgical aspects.

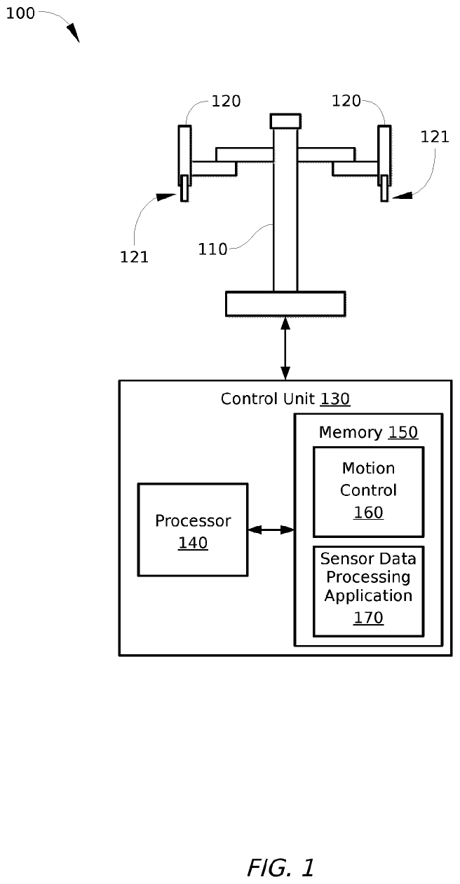

FIG. 1 is a simplified diagram of a computer-assisted system 100 according to some embodiments. As shown in FIG. 1, computer-assisted system 100 includes a device 110 with one or more kinematic chains, each kinematic chain comprising one or more joints and one or more links. For each kinematic chain, the one or more joints and links may comprise a repositionable arm 120. Each repositionable arm is configured to support one or more end effectors 121, and each of the one or more repositionable arms may comprise a teleoperated manipulator. In some examples, device 110 may be consistent with a computer-assisted medical device, such as a computer-assisted surgical device, and the one or more repositionable arms 120 provide support for one or more manipulation instruments, imaging devices, and/or the like. In some embodiments, computer-assisted surgical devices with other configurations, fewer or more kinematic chains, and/or the like may be used with computer-assisted system 100.

In some embodiments, the device 110 may operate on an object located on a table, and be mounted near or adjacent a workspace or the table, be mounted directly to the table, or be mounted to a rail coupled to the table, or be integrated as part of the table structure. In medical examples, the table may be an exam table, a surgical table, etc. In some embodiments, the device 110 may be a movable cart (e.g., a patient-side cart in a medical example). The movable cart may be separate from and spaced from any tables and may be independently movable relative to such tables. In some embodiments, the movable cart may be docked or attached to a table. In some embodiments, the device 110 may be mounted to a ceiling, floor, and/or wall of a room. In some embodiments having a plurality of devices 110, each device may be mounted to any structure or located in any manner as described above. For example, one device 110 may be mounted to a surgical table and another device 110 may be mounted to a ceiling.

Device 110 may further be coupled to an operator workstation (not shown), which may include one or more master controls for selectively operating device 110, the one or more repositionable arms 120, and/or the end effectors. The master controls are input devices that enable an operator to manipulate end effectors 121 and, in some embodiments, repositionable arms 120. Specifically, as the operator performs a procedure by manipulating one or more master controls (not shown), control unit 130 manipulates a respective repositionable arm 120 and/or end effector 121. In some embodiments, device 110 the operator workstation, and the control unit 130 may correspond to a da Vinci® Surgical System commercialized by Intuitive Surgical, Inc. of Sunnyvale, California.

Device 110 is coupled to a control unit 130 via an interface. The interface may include one or more cables, connectors, and/or buses, and may further include one or more networks (e.g., wired and/or wireless networks) with one or more network switching and/or routing devices. Control unit 130 includes a processor 140 coupled to memory 150. Operation of control unit 130 is controlled by processor 140. And although control unit 130 is shown with only one processor 140, it is understood that processor 140 may be representative of one or more central processing units, multi-core processors, microprocessors, microcontrollers, digital signal processors, field programmable gate arrays (FPGAs), application specific integrated circuits (ASICs), tensor processing units (TPUs), and/or the like in control unit 130. Control unit 130 may be implemented as a stand-alone subsystem and/or board added to a computing device or as a virtual machine. In some embodiments, control unit may be included as part of the operator workstation and/or operated separately from, but in coordination with the operator workstation.

Memory 150 may be used to store software executed by control unit 130 and/or one or more data structures used during operation of control unit 130. Memory 150 may include one or more types of machine-readable media. Some common forms of machine-readable media may include floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, and/or any other medium from which a processor or computer is adapted to read.

As shown, memory 150 includes a motion control application 160 that may be used to support autonomous and/or semiautonomous control of device 110. Motion control application 160 may include one or more application programming interfaces (APIs) for receiving position, orientation, motion, and/or other sensor information from device 110, exchanging position, orientation, motion, and/or collision avoidance information with other control units regarding other devices, such as a surgical table and/or imaging device, and/or planning and/or assisting in the planning of motion for device 110, repositionable arms 120, and/or end effectors 121 of device 110. And although motion control application 160 is depicted as a software application, motion control application 160 may be implemented using hardware, firmware, software, and/or a combination thereof, any of which interact with or are otherwise executed by processor 140.

In some embodiments, memory 150 further includes a sensor data processing application 170 that is configured to determine joint states, computer-assisted device modes, operator interaction details, environmental conditions, and the link. As specific examples, sensor data processing application 170 may comprise an operator interaction analysis application. The operator interaction analysis application is configured to determine, using at least sensor data comprising information about the operator interaction, location information of the operator interaction such as contact location(s), the type of operator interaction, the timing of the operator interaction, the sizes and/or shapes of contacts, etc. As another specific example, sensor data processing application 170 may comprise an image analysis application configured to determine, using at least images represented by image sensor data, the location and/or orientation of specified objects in or around device 110. In some embodiments, such an operator interaction analysis application or computer vision application includes one or more artificial intelligence-based algorithms for determining the relevant information using sensor data.

In some medical embodiments, computer-assisted system 100 may be found in an examination room, an operating room, and/or an interventional suite. Although FIG. 1 depicts computer-assisted system 100 with one device 110 having two kinematic chains and two repositionable arms 120, one of ordinary skill would understand that computer-assisted system 100 may include any number of devices, with each device having one or more kinematic chains comprising repositionable arm(s) 120 configured to support end effector(s) 121 of similar and/or different design from device 110. In some examples, each of the devices may include fewer or more kinematic chains, repositionable arms, and/or end effectors 121. Further, a computer-assisted system that includes one or more repositionable arms 120 can be configured with a different general architecture than that illustrated for computer-assisted system 100 in FIG. 1. In some embodiments, one or more portions of control unit 130, processor 140, memory 150, motion control application 160, and/or sensor data processing application 170 may be located on one or servers and/or cloud computing devices.

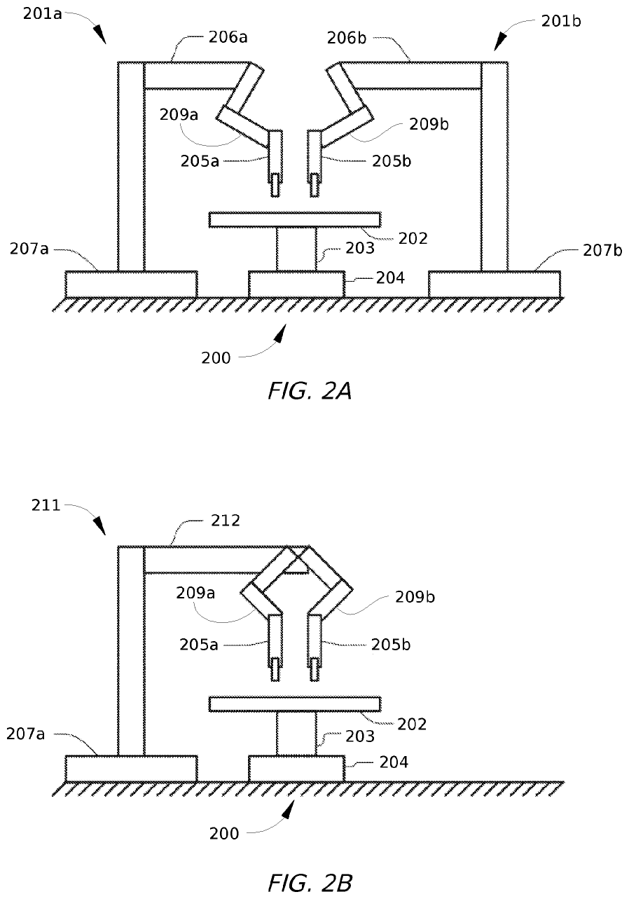

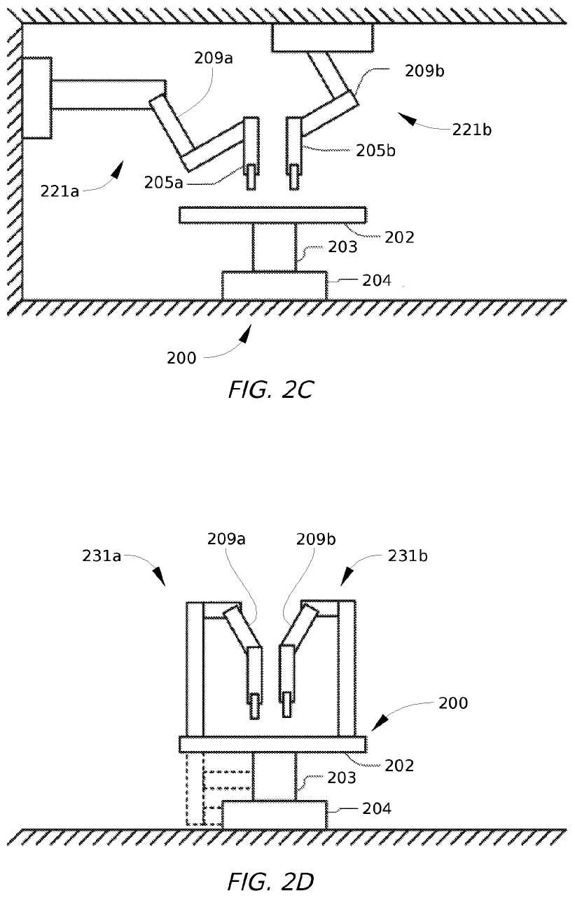

FIGS. 2A-2D are simplified schematic views that illustrate various computer-assisted device system architectures for a computer-assisted device with one or more repositionable arms 120, according to various embodiments.

FIG. 2A schematically illustrates a table 200 and a computer-assisted device 201a, according to an embodiment. Table 200 includes a table top 202 and a table support structure 203 that extends from a table base 204 to support the table top 202. Computer-assisted device 201a includes a kinematic chain coupled to a column. The kinematic chain comprises the links and joints of support structure 206a, and the repositionable arm 209a supported by support structure 206a. The repositionable arm 209 is configured to couple with an instrument assembly 205a. The support structure 206a is mechanically coupled at a proximal base 207a. The kinematic chain comprising support structure 206a and repositionable arm 209a enables movement and holding of instrument assembly 205a in various poses.

FIG. 2A further shows an optional second computer-assisted device 201b, which illustrates that two, three, four, five, or more individual computer-assisted devices may be included in a computer-assisted system. Each computer-assisted device may comprise one or more kinematic chains, with each kinematic chain having a corresponding repositionable arm.

As shown in FIG. 2A, second computer-assisted device 201b comprises a second kinematic chain coupled to a second column. This second kinematic chain comprises one or more links and one or more joints. The one or more links and joints of the second kinematic chain comprise a second support structure 206b and a second repositionable arm 209b. Second repositionable arm 209b is configured to support a second instrument assembly 205b. Second computer-assisted device 201b can hold and pose instrument assembly 205b using its kinematic chain. Computer-assisted devices 201a and 201b together comprise a computer-assisted system, and may operate together to perform a task. In some examples, computer-assisted devices 201a and/or 201b may be consistent with computer-assisted system 100 in FIG. 1, and repositionable arms 209a of computer-assisted device 201a and/or repositionable arms 209b of computer-assisted device 201b operate as disclosed herein.

FIG. 2B schematically illustrates table 200 and a computer-assisted device 211, according to an embodiment. Computer-assisted device 211 comprises a kinematic chain with one or more links and one or more joints. The one or more links and joints comprise a combined support structure 212 supporting two repositionable arms 209a and 209b. Repositionable arms 209a and 209b are configured to support instrument assemblies 205a and 205b. Computer-assisted device 211 illustrates that a computer-assisted device (including the various computer-assisted devices shown in FIGS. 2A-2D) may comprise a plurality of repositionable arms such as two, three, four, five, or more repositionable arms; each repositionable arm is configured to support an instrument assembly. In the embodiment illustrated in FIG. 2B, moving combined support structure 212 moves both repositionable arms 209a and 209b, and thus can move both instrument assemblies 205a and 205b together as a group. Repositionable arms 209a and 209b can also be moved separately to separately move instrument assembly 205a and 205b. Examples of such multi-repositionable arm devices include the DA VINCI SI, XI, X, and SP Surgical Systems commercialized by Intuitive Surgical, Inc. In some examples, computer-assisted device 211 is consistent with computer-assisted system 100 in FIG. 1, and repositionable arms 209a and/or 209b of computer-assisted device 211 operate as disclosed herein.

The computer-assisted devices of FIGS. 2A and 2B are each shown disposed on the floor. Alternatively, one or more such computer-assisted devices may optionally be located at a wall or ceiling, and be permanently fixed or movable with reference to such a wall or ceiling. In some examples, computer-assisted devices may be mounted to the wall or ceiling using a track or grid system that allows the support base of the computer-assisted devices to be moved. In some examples, one or more fixed or releasable mounting clamps may be used to mount the respective support bases to the track or grid system. As shown in FIG. 2C, a computer-assisted device 221a is mounted at a wall and a computer-assisted device 221b is mounted at a ceiling. Computer-assisted device 221a and computer-assisted device 221b may operate together as a system, or operate separately. Repositionable arms 209a of computer-assisted device 221a and/or repositionable arms 209b of computer-assisted device 221b operate as disclosed herein.

In some embodiments, computer-assisted devices may be mechanically coupled to a table such as table 200. As shown in FIG. 2D, a computer-assisted device 231a is coupled to the table top 202 of table 200. Computer-assisted device 231a may optionally be coupled to other portions of table 200, such as table support structure 203 or table base 204, as indicated by the dashed structures shown in FIG. 2D. In some implementations, the table 200 is movable, and movement of the portion of the table 200 attached to computer-assisted device 231a moves computer-assisted device 231a. FIG. 2D also shows a second computer-assisted device 231b that optionally may be present to create a multi-repositionable arm system. Repositionable arm 209a of computer-assisted device 231a and/or repositionable arm 209b of computer-assisted device 231b operate as disclosed herein.

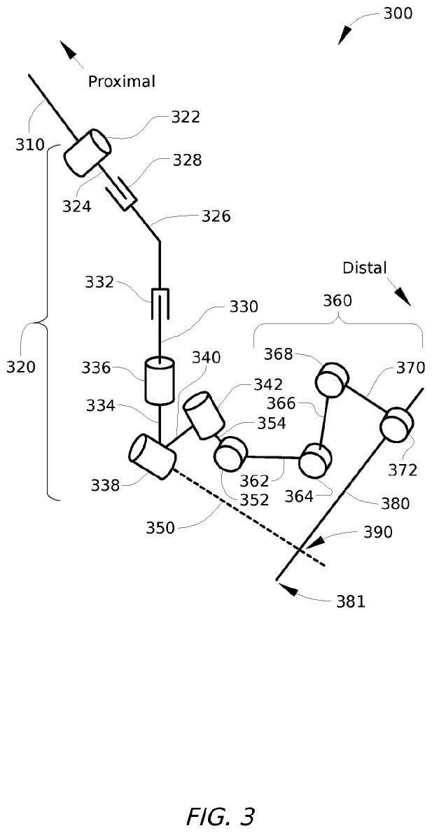

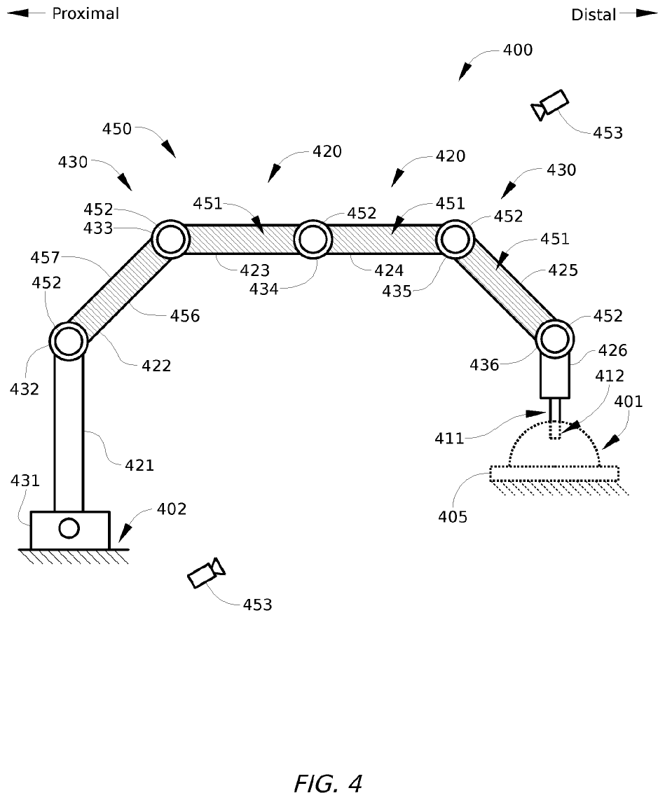

FIG. 3 is a simplified diagram showing a kinematic chain with one or more links and one or more joints, where the one or more links and joints comprise a repositionable arm 300, according to some embodiments. In some embodiments, repositionable arm 300 may be consistent with one of the repositionable arms 120 or a portion of one of the repositionable arms 120 of FIG. 1. In other embodiments, repositionable arm 300 may be consistent with one of the repositionable arms 209a or 209b or a portion of one of the repositionable arms 209a or 209b of any of FIGS. 2A-2D. Similarly, the kinematic chain of FIG. 3 may be consistent with the kinematic chains corresponding to any of FIGS. 1, 2A-2D.

In the example shown in FIG. 3, the repositionable arm 300 comprises a kinematic chain with a plurality of links coupled by a plurality of joints, and the plurality of links and joints comprise a repositionable arm. The most proximal end of repositionable arm 300 is coupled to a platform 310. In some examples, platform 310 may be coupled to additional joints and links (not shown) located proximal to platform 310. Coupled to platform 310 is a series of set-up joints and links 320 of the repositionable arm. The set-up joints and links 320 are rotationally coupled to platform 310 via a first set-up joint 322. In some examples, additional set-up links and joints for other repositionable arms (not shown) may be coupled to platform 310. Coupled to the first set-up joint 322 is a set-up base link 324 that is coupled to a proximal end of a set-up extension link 326 via a first set-up prismatic joint 328. A distal end of the set-up extension link 326 is coupled to a proximal end of a set-up vertical link 330 via a second set-up prismatic joint 332. A distal end of the set-up vertical link 330 is rotationally coupled to a proximal end of a support link 334 via a second set-up joint 336. Repositionable arm further comprises a first rotational joint 338 coupled to a distal end of the support link 334. The first rotational joint 338 provides rotational control over the additional links and joints located distal to the first rotational joint 338. In some examples, a central axis 350 of the first rotational joint 338 may be aligned with a remote center of motion 390 that may be fixed in location during teleoperation of the repositionable arm 300.

The repositionable arm 300 of the kinematic chain shown in FIG. 3 comprises further links and joints. A coupling link 340 couples the first rotational joint 338 to a second rotational joint 342. The second rotational joint 342 is coupled to a third rotational joint 352 via a link 354. Coupled distally to the third rotational joint 352 are additional links 362, 366, 370 coupled by additional rotational joints 364, 368, 372. An instrument coupling interface is located distally to rotational joint 372. An instrument 380 is shown coupled to repositionable arm 300. One or more end effectors (not shown in FIG. 3) may be coupled to a distal portion 381 of the instrument 380.

As shown in FIG. 3, the kinematic chain comprising repositionable arm 300 includes numerous links 324, 326, 330, 334, 340, 354, 362, 366, and 370 whose relative positions and/or orientations may be adjusted using numerous prismatic joints 328 and 332 and/or numerous rotational joints 322, 336, 338, 342, 352, 364, 368, and 372. Each of the prismatic and rotational joints may be associated with one or more sensors for sensing position, rotation, movement, force, torque, and/or the like on the respective joints.

Depending upon the design of the kinematic chain comprising repositionable arm 300, each of the plurality of joints of the kinematic chain may be a non-actuated joint or an actuated joint. In some examples, a non-actuated joint may not include any actuators, or may include only actuator(s) with insufficient motive power to move the associated joint, and therefore is not capable of causing motion via teleoperation and/or motion control commands from a control unit for repositionable arm 300. In some examples, the non-actuated joint may include a brake that permits the control unit to prevent and/or restrict motion in the non-actuated joint. In the example embodiment illustrated in FIG. 3, joints 328, 332, and/or 336 may be non-actuated joints. In some examples, an actuated joint may include one or more actuators that may control motion of the actuated joint, and may be commanded to move the joint teleoperatively and/or carry out other motion commands. In some examples, an actuated joint may further include a brake. In such examples, the brake may be employed in an actuated joint to hold a current pose of the non-actuated joint rather than to actively control motion of the actuated joint. In some embodiments, the brake employed in an actuated joint and/or a non-actuated joint is configured to operate in a binary fashion. In some embodiments, when the brake is activated, the brake fully engages and applies a specified braking force that holds the joint in a current position. In some embodiments, when the brake is deactivated, the brake fully disengages and applies very little or no braking force. In some embodiments, the brake employed in an actuated joint and/or a non-actuated joint is configured to operate with variable friction. In some embodiments, the variable-friction brake is configured to apply controllable friction when the brake is activated, where the controllable friction can be varied continuously from a fully engaged braking value to fully a released braking value.

According to various embodiments, a set of joints in the kinematic chain of a computer-assisted device is placed into a floating state. In some embodiments, one or more joints of the computer-assisted device are placed into the floating state based on one or more parameters, including location information of an operator interaction. Location information of such operator interaction may be determined via any appropriate sensing technology. Example sensors include: image sensors including optical sensors, acoustic sensors, electromagnetic sensors, proximity or presence sensors, contact sensor, pressure sensors, force and torque sensors, position sensors, velocity or speed sensors, buttons and switches, etc. The sensors can be configured to sense the operator interaction directly (such as by sensing the force of a touch by an operator) or indirectly (such as by sensing a joint parameter change due to an operator interaction with a link coupled to the joint). Example joint parameters include joint position, joint velocity, joint force or torque, etc. Sensors may be disposed on, in, or outside of the joints, links, or other components of the kinematic chain, as appropriate for the sensing technology. As described earlier, the one or more joints that are placed into a floating state in response to the operator interaction may be determined based on various parameters. Example parameters include the type of interaction, such as if the interaction comprises grasping, pushing, touching, tapping. Example parameters also include interaction location(s) to any appropriate level of resolution, such as which link or joint is experiencing the operator interaction, or which location on the link or joint the operator interaction is being applied. Additional examples of parameters include: a number of locations of the operator interaction (e.g., one, two, three, four, etc. contact locations); the current task being performed using the computer-assisted device; a current pose of the kinematic chain; a current state of the computer-assisted device; a type of joint adjacent to a link being interacted with by the one or more operators; a type of joint located between links interacted with by the one or more operators, whether the kinematic chain is subject to a constraint, a direction of an asserted force associated with the operator interaction, and/or the like as is described in further detail below.

FIGS. 1-3 set forth non-limiting examples of computer-assisted systems, computer-assisted devices, and kinematic chains, and various embodiments may differ in construction. For example, a computer-assisted system may comprise any number of computer-assisted devices, a computer-assisted device may comprise any number of kinematic chains, and a kinematic chain may comprise any number of repositionable arms. Further, a kinematic chain may comprise more or fewer joints and links than shown in FIGS. 1-3, and be of different sizes, shapes, and structures than those shown in FIGS. 1-3; as a specific example, FIGS. 2A-2D show kinematic chains with a support structure comprising a single link, and other embodiments may comprise support structures with any number of links or joints, or no support structure at all. In addition, although FIGS. 1-2D show kinematic chains coupled to columns, and FIG. 3 shows a kinematic chain coupled to a platform, kinematic chains may be coupled to other components, such as to bases, to additional kinematic chains, etc.

Also, a kinematic chain may have redundant degrees of freedom, or no redundant degrees of freedom. A kinematic chain with redundant degrees of freedom can use multiple different configurations of the joints of the kinematic chain to achieve a same position, or a same position and orientation, for a portion of interest of the kinematic chain. As a specific example, a computer-assisted device may comprise a kinematic chain with redundant actuated degrees of freedom; such a computer-assisted device may command motion of the kinematic chain such that a first portion of the kinematic chain moves while a position, or a position and orientation, is maintained for a second portion of the kinematic chain. U.S. Pat. No. 8,749,190 contains a description of an example computer-assisted device comprising a kinematic chain with redundant degrees of freedom that are actuated.

Various examples of kinematic chains are described below in conjunction with FIGS. 4 and 6-10. However, FIGS. 4 and 6-10 are provided as non-limiting examples that do not limit the scope of the various embodiments described further herein. For example, the various embodiments described further herein can be applied to kinematic chains different from those illustrated in FIGS. 4 and 6-10, different types and/or locations of operator interaction are possible, different parameters can be used to determine which joints to place in the floating state, different types of constraints on the kinematic chains are possible, and/or the like. As a specific example, FIGS. 4 and 6-10 show kinematic chains with redundant degrees of freedom; however, various embodiments include kinematic chains with no redundant degrees of freedom.

Placing a joint of a kinematic chain in a floating state facilitates changes in the joint position of the floated joint, and thus facilitates the reconfiguration of the kinematic chain. A joint that is held in position by a brake can be placed into a floating state by releasing the brake partially or entirely. A partial release may be used, for example, to help the joint stay in place against gravity, to provide some structural stability as the joint is externally manipulated, to provide damping, etc. A joint that is held in position by an actuator may be placed into a floating state by updating the actuator commands to the current position of the joint; with this approach, external deflection of the joint will provide a new commanded position for the joint.

A joint that is held in position by both a brake and an actuator can be placed into a floating state by a combination of brake release and updated actuator commands. The floating state enables an operator to manually change the positions and/or the orientations of certain portions of the kinematic chain via direct manipulation. A joint in the floating state may exit the floating state and enter a non-floating state in response to one or more exit conditions.

Example exit conditions include: a passage of a predetermined period of time, an cessation of an operator interaction, a cessation of external manipulation of the joints in the floating state or the kinematic chain, a movement not adhering to a constraint to which the kinematic chain is subject, an operator input to exit the floating state, etc. A description of the configuration and operation of joints that can be placed into a floating state under certain conditions, and to be placed into a non-floating state (in this example, a locked state) under other conditions can be found in U.S. Pat. No. 10,489,008, which is incorporated in its entirety herein.

FIG. 4 is a schematic diagram of a kinematic chain 400 configured according to some embodiments. In some embodiments, kinematic chain 400 may be included in and consistent with any kinematic chain described herein, including those described in conjunction with any of FIGS. 1-3. As shown, kinematic chain 400 includes a plurality of links (referred to collectively herein as links 420) and a plurality of joints (referred to collectively herein as joints 430) that are configured to position and orient an end effector at the end of shaft 411 in various configurations. In some embodiments, the end effector 412 at the end of shaft 411 is consistent with one of end effectors 121 in FIG. 1 and/or one of instrument assemblies 205a or 205b in FIGS. 2A-2D.

As shown in FIG. 4, links 420 include links 421-426 and joints 430 include joints 431-436. Joint 431 is coupled to a stationary base 402, which is disposed at the proximal end of kinematic chain 400. Link 426 is coupled to end effector 412 via shaft 411, which is disposed at the distal end of kinematic chain 400. For simplicity, joints 431-436 are depicted as joints that are configured with a single degree of freedom, e.g., rotation about an axis, and oriented to allow motion of links 421-426 in a single two-dimensional plane. However, in some embodiments, joints 431-436 can be configured with multiple degrees of freedom and may be oriented to allow motion of one or more of links 421-426 in three-dimensional space. In some embodiments, one or more of joints 431-436 may be configured as a rotational joint having a single degree of freedom (e.g., rotation) allowing the rotational joint to move about a single axis of rotation. In some embodiments, one or more of joints 431-436 may be configured as a prismatic joint having a single degree of freedom (e.g., translation) allowing the prismatic joint to move along a single axis of linear motion. In some embodiments, one or more of joints 431-436 may be configured with multiple degrees of freedom allowing the respective joints to move about each of the multiple degrees of freedom. In the example illustrated in FIG. 4, end effector 412 at the end of a shaft 411 has been inserted through a port in object 401 to access a workspace in object 401.

In some instances, motion of kinematic chain 400 is limited by the degrees of freedom and ranges of motion defined by the structure of kinematic chain 400. In some instances, motion of kinematic chain 400 is further limited by a constraint imposed on kinematic chain 400, for example the positioning of end effector 412 with respect to object 401. In some embodiments, a constraint may limit the movement of a constrained portion of kinematic chain 400 in one or more degrees of freedom. For example, a constraint may limit the constrained portion in translation in one or more directions, rotation about one or more axes, or in both translation and rotation. As a specific example, a constraint may limit the constrained portion to stay fixed in space in both position and orientation. A constraint can be physically imposed by a physical element (e.g. where an obstacle physically impedes motion of a portion of kinematic chain 400), virtually imposed by software-imposed limits, such as calculated limits, on position or motion (e.g. where the constraint limits commanded motions to hold a particular portion static in position and/or orientation), or a combination physical and virtual constraint (e.g. where an obstacle impedes some degrees of freedom and calculated limits impedes other degrees of freedom). A constraint may be imposed distally relative to a contact location of an operator interaction, at a contact location, or proximally relative to the contact location. A computer-assisted device comprising a kinematic chain can determine which joint(s) to place in a floating state further based on any applicable constraints. In some embodiments, a constraint on the position of a distal portion of kinematic chain 400 causes one or more distal elements of kinematic chain 400 to remain substantially stationary in space even when one or more joints of that kinematic chain are in a floating state.

In some embodiments, kinematic chain 400 is monitored using a sensor system of one or more sensors (referred to collectively herein as sensors 450) to facilitate determining operator interactions. In some embodiments, the sensor system may facilitate determining location information. In some embodiments, the sensor system may facilitate determining which of links 421-426 or joints 431-436 is interacted with by the one or more operators, the number of locations of the operator interaction, the type of operator interaction, the current task being performed using the computer-assisted device, the current pose of the kinematic chain; the current state of the computer-assisted device, whether the kinematic chain is subject to a constraint, a direction of an asserted force associated with the operator interaction, and/or the like. As noted above, a computer-assisted device may, based on one or more such determinations, place one or more of joints 431-436 in a floating state.

In some embodiments, link-based sensors 451 configured to detect interactions with one or more of links 421-426 are utilized. In some embodiments, link-based sensors 451 can generate one or more signals that enable determination of location information, how the one or more operators are interacting with a particular link of links 421-426, or any of the other determinations discussed above. In some embodiments, data from link-based sensors 451 can be used to determine if an operator interaction has occurred, and if so, the location(s) or type(s) of operator interaction. As a specific example, in some embodiments, detection of operator contact on opposite sides 456 and 457 of link 422 indicates a grab-type interaction by an operator with link 422. As another specific example, in some embodiments, a shape of an area of contact, such as one provided by a higher-resolution capacitive, temperature, or pressure sensor matching grab areas of contact indicates a grab-type interaction. In some embodiments, link-based sensors 451 can indicate direction of asserted force on one or more of links 421-426, for example with respect to a surface experiencing the interaction, including if the force comprises tangential components, normal components, or both tangential and normal components, relative to the surface.

In some embodiments, data from the sensor system can be used to differentiate between an accidental and a deliberate input. For example, in some embodiments, link-based sensors 451 provide a shape of the operator interaction contacting a link of links 421-426, and that shape can be analyzed to determine if the shape sufficiently matches a hand (instead of some other body part) or a grab-interaction (instead of a bump or some other interaction). In some embodiments, images from image sensors, force profiles from force sensors, contact profiles from capacitive or pressure sensors, etc. can be processed by trained neural networks to determine if the interaction is deliberate or accidental.

In some embodiments, joint-based sensors 452 configured to detect interactions with one or more of joints 431-436, or to detect one or more joint parameters, are utilized. Example joint parameters include: a joint position, joint velocity, joint acceleration, and joint force or torque. For example, data from joint-based sensors 452 can be used to determine joint position, which can be used to determine a kinematic chain configuration. As another example, data from joint-based sensors 452 can be used to determine joint deflection which can be used to determine locations, directions, or magnitudes of forces or torques associated with operator interactions.

In some embodiments, environmental sensors 453 are utilized. Examples include image sensors such as cameras that capture images of the kinematic chain 400 and the surrounding environment. In some embodiments, environmental sensors 453 may provide sufficient situational information to enable semantic decomposition, in real-time, of instances in which an operator interaction occurs between an operator and kinematic chain 400. In some embodiments, sensor data processing application 170 of FIG. 1 may be configured to perform such semantic decomposition based on situational information generated by environmental sensors 453. For instance, in some embodiments, such situational information can include movement by the operator relative to kinematic chain 400. In some examples, automated motion of one or more portions of kinematic chain 400 that result in contact with an operator may be interpreted, by an image processing application of the sensor data processing application 170, to be unintended contact between kinematic chain 400 and the operator. In some examples, orientation of an operator relative to the portion of kinematic chain 400 contacted can indicate whether the detected contact between kinematic chain 400 and the operator is intentional. In some examples, contact by certain body parts of an operator against the portion of kinematic chain 400 contacted can indicate whether the detected contact between kinematic chain 400 and the operator is intentional. For instance, use of a hand and/or arm by an operator may indicate intentional contact, while contact from another portion of the body of the operator may indicate unintentional contact.

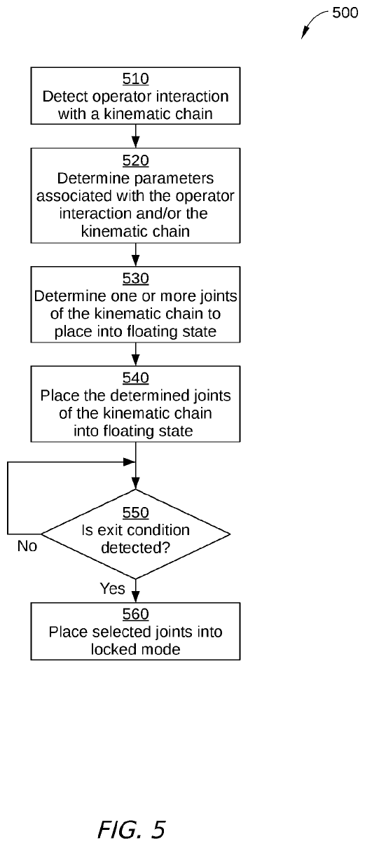

FIG. 5 is a simplified diagram of a method 500 of placing joints of a computer-assisted system into a floating state, according to some embodiments. One or more of the processes 510-560 of method 500 may be implemented, at least in part, in the form of executable code stored on non-transient, tangible, machine readable media that when run by one or more processors (e.g., the processor 140 in control unit 130) may cause the one or more processors to perform one or more of the processes 510-560. In some embodiments, the method 500 may be performed by an application, such as motion control application 160 and/or sensor data processing application 170. According to some embodiments, method 500 can include more or fewer processes than those depicted in FIG. 5. In some embodiments, one or more of processes 550 and/or 560 are optional and may be omitted.

At a process 510, an operator interaction with kinematic chain 400 is detected. In some embodiments, one or more signals are received from one or more sensors 450, indicating one or more operator interactions with kinematic chain 400. In some embodiments, the signals are received from sensors of a sensor system. For example, the sensor system may comprise one or more link-based sensors 451, one or more joint-based sensors 452, one or more environmental sensors 453, and/or other sensors. Such signals can indicate location information about the operator interaction(s), such as the number of contacts or the contact location(s) of each operator interaction. In some embodiments, such signals can indicate a magnitude and direction of force of an operator interaction; the type of object providing an operator interaction (e.g. a tool manipulated by the operator, an operator body part such as fingers, a hand, a forearm, a torso, or some other body part, whether the body part is bare or gowned, etc.); a number of contacts for an operator interaction, the size or shape of contact(s) of an operator interaction; a type of operator interaction, and/or the like. In some embodiments, such signals can also indicate a resultant magnitude and direction of force exerted on a joint or a link due to an operator interaction, a resultant magnitude and direction of torque exerted on a joint or a link due to an operator interaction, and/or the like. In some embodiments, such signals can also provide contextual information related to an operator interaction, and be used to determine whether the operator interaction is more likely to be an intentional operator interaction, an unintentional operator interaction (e.g. accidental contact), or the like. In some embodiments, the operator interaction may be associated with interactions between kinematic chain 400 and a single operator or two or more operators.

At a process 520, one or more parameters are determined. The one or more parameters may comprise parameters associated with the operator interaction detected in process 510, the operating environment, and/or the computer-assisted system (e.g. the kinematic chain 400).

In some embodiments, the one or more parameters determined in process 520 include location information, and are determined based on the signals received from one or more sensors 450 during process 510. Thus, location information for an operator interaction with kinematic chain 400 is determined. In some embodiments, the location information includes which portion or portions of the kinematic chain 400 are receiving the operator interaction, one or more contact locations of the detected operator interaction to any appropriate resolution, etc. In some embodiments, the location information includes specific locations of kinematic chain 400 that are being interacted with by an operator. In some embodiments, the signals received from one or more sensors 450 are used to determine parameters other than location information. Example other parameters include: the type of operator interaction, the duration of an operator interaction, instantaneous or historical forces associated with the operator interaction, a number of operator interactions, etc. In some embodiments, the one or more parameters determined in process 520 include force magnitude and direction information associated with the operator interaction and/or torque magnitude and direction information associated with the operator interaction.

In some embodiments, the one or more parameters include one or more of the following: the current task being performed using the computer-assisted device; a current pose of kinematic chain 400; the type of joint adjacent to a link being interacted with by the operator interactions; the type of joint located between links interacted with by the operator interaction; whether the kinematic chain is subject to a constraint in one or more degrees of freedom and details of the constraint; a direction of an asserted force associated with the operator interaction; a number of contact locations of the operator interaction; a type of each of the one or more operation interactions that is detected (intentional or unintentional, push or pull, touch or grab, and the like); a type of joint that is adjacent to a contact location of a detected operator interaction; one or more joint parameters; an operating mode of the computer-assisted system; a functional status of the computer-assisted system; and/or the like. Some of the parameters are determined partially or entirely from data other than sensor signals. For example, in some embodiments, an operating mode of the computer-assisted system is determined from a state variable stored and updated by the computer-assisted system as it operates.

The parameters can also be combined to provide a more complete description of the operator interaction, such as which type of operator interaction is occurring at which contact locations during what operating mode of the computer-assisted system, for that particular operator interaction.

In some embodiments, the one or more parameters determined in process 520 include a current state of the computer-assisted device and/or the kinematic chain. Example states of the computer-assisted device include: being in a setup state; being in a fault state; having one or more kinematic links in motion; having no tools or particular tools installed; and (in a medical example) being in a curtained or draped sterile operating area; and/or the like.

At a process 530, one or more joints of kinematic chain 400 to be placed into the floating state are determined. The joints 431-436 (if any) to be placed into the floating state may be based on one or more of the parameters determined during process 520, such as based on location information of a first operator interaction with the kinematic chain 400.

In some examples, a configurable number of the closest joints 431-436 that are proximal to a single operator interaction are placed into the floating state. In some examples, a single joint of joints 431-436 that is proximal to the single operator interaction is placed into the floating state. In some examples, a configurable number of the closest joints 431-436 that are proximal to a single operator interaction are placed into the floating state. In some examples, at least one of joints 431-436 that is distal to the location of operator interaction is placed into the floating state. In some examples, some or all of joints 431-436 that are disposed between a first contact location of a first operator interaction and a second contact location of a second operator interaction are placed into the floating state. In some examples, a single one of joints 431-436 that is proximal to the first contact location and the second contact location is placed into the floating state.

In some embodiments, the one or more parameters for determining which of joints 431-436 to select in process 530 include the location or locations of the operator interactions, the number of contacts in an operator interaction, and/or the number of operator interactions detected in process 510. In some examples, an operator interacts with kinematic chain 400 via a pushing or pulling action against a particular link 421-426 (or a particular joint 431-436). In response, one or more of joints 431-436 are placed into a floating state, so that motion of such joints and one or more of links 421-426 in contact with such joints is facilitated. As a result, further operator interaction (e.g. further pushing or pulling, etc. against any of the links 421-426) externally manipulates at least a portion of kinematic chain 400 from an initial configuration to a subsequent configuration. In such examples, one or more of joints 431-436 that are placed into the floating state may include some or all joints 431-436 that are proximal to a single operator interaction; a configurable number of the closest joints 431-436 that are proximal to the single operator interaction; a single one of joints 431-436 that is proximal to the single operator interaction; some or all of joints 431-436 that are disposed between a first contact location of a first operator interaction and a second contact location of a second operator interaction; a single one of joints 431-436 that is proximal to the first contact location and the second contact location; and the like.

In some embodiments, the one or more parameters for determining which of joints 431-436 to select in process 530 include the type of operator interactions detected in process 510 (e g , intentional or unintentional, push or pull, touch or grab, and the like). In some examples, when an interaction is not determined to be an intentional operator interaction, none of joints 431-436 are placed into a floating state, such as when the interaction is by an object instead of by a body part of an operator, when a gowned portion of the body of an operator is determined to have collided with a portion of kinematic chain 400, or when the operator interaction is determined to be accidental.

In some embodiments, the one or more parameters for determining which of joints 431-436 to select in process 530 include the type of a joint 431-436 that is adjacent to a link from links 421-426 that is associated with a contact location of an operator interaction detected during process 510. In some examples, a set of one or more of joints 431-436 of kinematic chain 400 is selected that includes one or more actuated joints and no non-actuated joints. In some embodiments, a set of one or more of joints 431-436 of kinematic chain 400 is selected that includes one or more non-actuated joints that are not configured with a degree of freedom in a direction that can be affected by gravity. In some embodiments, one such non-actuated joint may have a single degree of freedom that only allows motion of one of links 421-426 perpendicular to the gravitational force vector. Thus, in some embodiments, when the non-actuated joint is placed into a floating state, a brake included in the non-actuated joint can be released without the possibility of gravity moving the link connected to the non-actuated joint. In some embodiments, a set of one or more of joints 431-436 of kinematic chain 400 is selected that does include one or more non-actuated joints that are configured with a degree of freedom in a direction that can be affected by gravity, and the joints are gravity compensated, dampened, partially braked, or otherwise configured to reduce the likelihood of gravity moving one or more of links 421-426 connected to the non-actuated joints in an unintended way.

In some embodiments, the one or more parameters for determining which of joints 431-436 to select in process 530 include a direction of force of a detected operator interaction in process 510. In some examples, a set of joints 430 of kinematic chain 400 is selected that includes one or more of joints 431-436 that enable motion in the direction of the detected force. In some embodiments when a direction of force is in a particular plane, one or more of joints 431-436 included in the set of joints 430 are joints that have a degree of freedom in that particular plane. In some embodiments, when the direction of force is in the particular plane, one or more of joints 431-436 included in the set of joints 430 are joints that, in combination, enable motion of a link of links 421-426 that is interacted with in the direction of force. In some embodiments, each joint in the set of joints 430 may be configured with a degree of freedom in a direction that includes a component of the direction of force. In some embodiments, actuation of a combination of one or more of joints 431-436 in the set of joints 430 enables motion of the link in the direction of force.

In some embodiments, the one or more parameters for determining which of joints 431-436 to select in process 530 include whether a constraint is imposed on a portion of kinematic chain 400. hi some examples, when a constraint is imposed on a portion of kinematic chain 400, some or all of joints 431-436 distal to a detected contact location remain in a locked state and are not placed into a floating state. In some examples, to further facilitate motion of a portion of kinematic chain 400, one or more of joints 431-436 that are proximal and/or distal to the detected operator interaction are driven to perform compensating motion that allows the kinematic chain 400 to adhere to the constraint even though one or more of joints 431-436 are in the floating state. In some examples, when the constraint is imposed on a portion of kinematic chain 400, a configurable number of the closest joints 431-436 that are proximal to the detected operator interaction are placed into a floating state. In some examples, when the constraint is imposed on a portion of kinematic chain 400, some or all of joints 431-436 that are proximal to the detected operator interaction are placed into the floating state.

In some embodiments, the one or more parameters for determining which of joints 431-436 to select in process 530 include whether no constraint being imposed on kinematic chain 400. In some examples, when no constraint is imposed on kinematic chain 400, one or more of joints 431-436 that are proximal to an operator interaction is placed into the floating state. In some examples, when no constraint is imposed on kinematic chain 400, the most proximal joint of joints 431-436 of kinematic chain 400 is placed into the floating state.

In some embodiments, the one or more parameters for determining which of joints 431-436 to select in process 530 include a current state of kinematic chain 400 and/or the computer-assisted device that includes kinematic chain 400, when an operator interaction is detected in process 510. In some embodiments, a current state of kinematic chain 400, and/or the computer-assisted device that includes kinematic chain 400, includes being in a setup state. For example, in a setup state, kinematic chain 400, and/or the computer-assisted device that includes kinematic chain 400, does not perform operations or procedures with end effector 412. Therefore, in some examples, no distal constraint is imposed on kinematic chain 400 in such a state. In some examples, when kinematic chain 400 and/or the computer-assisted device that includes kinematic chain 400 is in a setup state when an operator interaction is detected, some or all of joints 431-436 that are proximal to the operator interaction are placed into the floating state.

In some embodiments, a current state of kinematic chain 400, and/or the computer-assisted device that includes kinematic chain 400, includes a motion status of at least of portion of kinematic chain 400. In some examples, when an operator interaction is detected during motion of the portion of kinematic chain 400, joints of joints 431-436 adjacent to a contact location of the operator interaction are placed into a floating mode. Thus, motion of kinematic chain 400 is modified to reduce impact of a collision with an operator or an object that is in an operating environment associated with the computer-assisted device. In some examples, to further facilitate motion of a portion of kinematic chain 400 associated with the contact location while allowing the kinematic chain 400 to adhere to the constraint, one or more of joints 431-436 that are proximal and/or distal to the detected operator interaction are driven to perform compensating motion. In some examples, some or all of joints 431-436 that are proximal to the operator interaction are placed into the floating state. In some examples, a configurable number of the closest joints 431-436 that are proximal to the single interaction are placed into the floating state. In some examples, a single joint of joints 431-436 that is proximal to the operator interaction is placed into the floating state. In some examples, at least one joint of joints 431-436 that is distal to the contact location is placed in the floating state.

In some embodiments, a current state of kinematic chain 400, and/or the computer-assisted device that includes kinematic chain 400, includes a current task or a current pose of kinematic chain 400, and/or of the computer-assisted device that includes kinematic chain 400. Examples of a current task include: being employed in a medical procedure while end effector 412 is inserted into a patient, moving end effector 412 to a particular location, performing a calibration procedure, and the like. In some examples, when an operator interaction is detected during such a task, or when kinematic chain 400 is in a certain pose, joints of joints 431-436 adjacent to a contact location of the operator interaction are placed into a floating mode. In some examples, to further facilitate motion of a portion of kinematic chain 400 associated with the contact location while the current task is being performed or the current pose of kinematic chain 400 is being maintained, one or more of joints 431-436 that are proximal and/or distal to the detected operator interaction are driven to perform compensating motion. In some examples, some or all of joints 431-436 that are proximal to the operator interaction are placed into the floating state. In some examples, a configurable number of the closest joints 431-436 that are proximal to the single interaction are placed into the floating state. In some examples, some or all of joints 431-436 that are disposed between a first contact location of a first operator interaction and a second contact location of a second operator interaction are placed into the floating state. In some examples, a single joint of joints 431-436 that is proximal to the first contact location and the second contact location are placed into the floating state. In some examples, a single joint of joints 431-436 that is proximal to the operator interaction is placed into the floating state. In some examples, at least one joint of joints 431-436 that is distal to the contact location is placed in the floating state.

At a process 540, the one or more of joints 431-436 determined to be ones to place into the floating state are placed into the floating state. In some embodiments, in the floating state a brake of a joint from joints 431-436 with a brake is partially or entirely released. In some embodiments, the brake is released partially to counteract gravitational force or to providing damping. In some embodiments, in the floating state an actuator of a joint of joints 431-436 held in position by the actuator, is commanded to actuate a joint of joints 431-436 to a current position; as the joint is moved by external manipulation, the commanded position is updated to the then-current position. In some embodiments, the actuator is also controlled to counteract gravitational force or to provide damping, while still facilitating joint movement in response to other external forces.

In some embodiments, while the one or more joints 431-436 are in the floating state the operator interaction detected during process 510, additional and/or other operator interactions with kinematic chain 400, and/or other external manipulations of kinematic chain 400, may cause the one or more of joints 431-436 to change in position. In some embodiments, one or more of joints 431-436 not in the floating state may be used to perform compensating motion to, for example, adhere to a constraint on kinematic chain 400. A detailed description of the configuration and operation of a repositionable arm with joints that can apply compensating motion to reduce motion of an end effector when proximal joints undergo motion can be found in U.S. Pat. No. 10,070,931, which is incorporated in its entirety herein.

At a process 550, an exit condition is detected. In some embodiments, the exit condition may correspond to movement below a threshold amount in the one or more of joints 431-436 in the floating state and/or movement below a threshold amount in the one or more of joints 431-436 for a predetermined period of time, such as is described in U.S. Pat. No. 10,034,717, which is incorporated in its entirety herein. In some embodiments, the exit condition may correspond to a predetermined period of time since the completion of process 540. In some embodiments, the exit condition may correspond to an operator command, a change in state of kinematic chain 400 or of the computer-assisted device, an error condition, and/or the like. If such an exit condition is not detected, process 550 repeats and the one or more of joints 431-436 remain in the floating state. If such an exit condition is detected, method 500 proceeds to a process 560.

At process 560, at least one joint of joints 431-436 in the floating state exits the floating state. In the example shown in process 560, these joints are placed into a locked state. In some embodiments, in the locked state for a joint of joints 431-436 with a brake, the brake is engaged to brake the joint and resist external manipulation. In some embodiments, in the locked state for a joint of joints 431-436 held in place by an actuator, the actuator is commanded to hold the position of the joint, such that motion of the joint is counteracted.

Method 500 may be repeated by returning to process 510 to handle additional operator interactions with kinematic chain 400. Further, in some embodiments, an additional operator interaction can be detected during method 500 that initiates a second method 500 for placing other joints of a kinematic chain into a floating state.

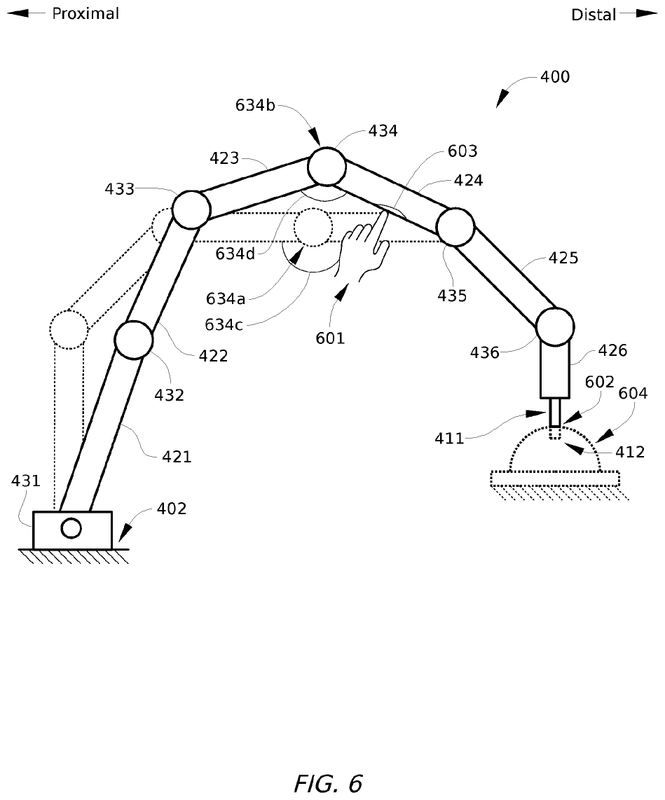

Example, non-limiting, use cases consistent with FIGS. 1-5 are described in more detail in FIGS. 6-10.

FIG. 6 schematically illustrates kinematic chain 400 responding to an operator interaction with a single contact location 603, according to some embodiments. As shown, an operator interacts with kinematic chain 400 via a pushing action 601 against link 424. In response, some of joints 431-436 are placed into a floating state, so that motion of such joints 431-436 and one or more links 421-426 in contact with such joints 431-436 is facilitated. FIG. 6 further illustrates how further operator interaction (e.g. further pushing against link 424 outward, pulling of link 423 outward, etc.) externally manipulates kinematic chain 400 from an initial configuration depicted by dotted-lines to a subsequent configuration illustrated by solid lines.

As shown in FIG. 6, a constraint 602 is imposed on kinematic chain 400. In the example of FIG. 6, end effector 412 at the end of shaft 411 has been inserted through a port to access a workspace in object 604, and the kinematic chain 400 has a constraint that limits the motion of the kinematic chain 400 such that shaft 411 does not move laterally relative to the port. The constraint limits the motion of shaft 411 to pivot about the location of the port, or to move in and out of the port. Thus, the constraint of FIG. 6 is located distally to pushing action 601. In some embodiments, constraint 602 may be consistent with the constraint imposed on kinematic chain 400 in FIG. 4, for example the positioning of end effector 412 with respect to object 401. In some embodiments, constraint 602 may be a software-imposed remote center of motion that constrains motion of a location offset from a distal link of kinematic chain 400, and be consistent with remote center of motion 390 described in FIG. 3. In a medical example, the constraint 602 could comprise a boundary based or static location (e.g. a point) where kinematic chain 400 interacts with a cannula inserted into patient anatomy or tissue.

In some embodiments, joints 431-436 that are placed into the floating state in response to operator interaction are selected to include some or all of joints 431-436 proximal to the link being contacted. For example, in the embodiment illustrated in FIG. 6, a set of joints 430 that are proximal to link 424 are determined to be placed into the floating state. In some embodiments, the set of joints 430 includes a configurable number (e.g., one, two, three, etc.) of the joints 431-436 closest to and proximal to link 424. In some embodiments, a set of joints 430 that are distal to link 424 is driven to perform compensating motion that facilitates motion of a portion of kinematic chain 400 associated with contact location 603 while allowing kinematic chain 400 to adhere to a constraint distal to the link 424, such as constraint 602.

In FIG. 6, the operator interaction at location 603 causes joint 434 to be placed into a floating state. A further pushing action at the same location 603 causes joint 434 to be displaced from original location 634a to a final location 634b. To enable the kinematic chain 400 to adhere to the constraint 602, joints 431, 432, 433, and 435 are driven to new positions that compensate for the motion of joint 434, such that shaft 411 stays within the constraint 602 associated with the port of object 604. Alternatively, others of joints 433, 432, and/or 431 may be placed in the floating state with one or more of joints 431-436 not in the floating state being using to provide the compensating motion to enable the kinematic chain 400 to adhere to the constraint 602. As shown in FIG. 6, the position of joint 434 has changed from an initial angle 634c between link 423 and link 424 to a final angle 634d. When joint 434 is switched out of the floating state and back to a non-floating state (which can be a locked state), such as when pushing action 601 and/or other operator interaction is determined to have stopped, joints 433 and 434 again hold themselves in a fixed pose. In some examples, when joint 433 or 434 is a non-actuated joint, the brake of the non-actuated joint is employed to hold the current pose of the joint, e.g., to compensate for gravitational force measured by a joint-based sensor 452. Alternatively, when joint 433 or 434 is an actuated joint, an actuator and/or a brake included in the actuated joint may be employed in the actuated joint to hold a current pose of the actuated joint.

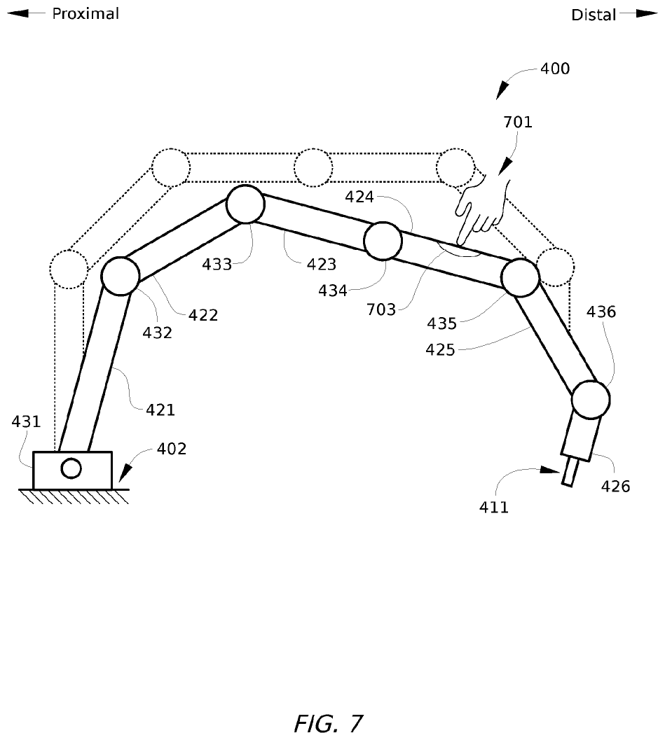

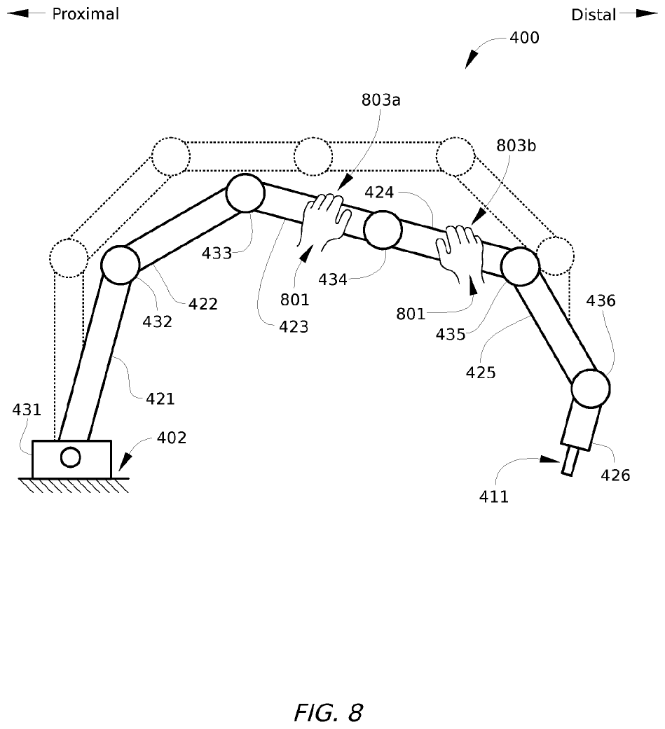

FIG. 7 schematically illustrates kinematic chain 400 responding to an operator interaction with a pushing action 701 at a single contact location 703 when no constraint is present, according to some embodiments. As shown, an operator interacts with kinematic chain 400 via a pushing action 701 against link 424. In response, some of joints 431-436 are placed into a floating state, so that motion of such joints and one or more of links 421-426 in contact with such joints is enabled. As shown in FIG. 7, the pose of kinematic chain 400 is not currently constrained by a constraint. In some embodiments, kinematic chain 400 may be in a setup state where the kinematic chain 400 has not been coupled to a port for access to a workspace, or end effector 412 of a tool coupled to the kinematic chain 400 is not engaged with a workspace.

In some embodiments, a specific joint of joints 431-436 proximal to single contact location 703 is placed into a floating state when pushing action 701 is detected at single contact location 703 and kinematic chain 400 is not currently constrained by a constraint. Thus, end effector 412 and some or all links 421-426 of kinematic chain 400 are moved by a continuing of pushing action 701 and/or a further operator interaction applied after the specific joint pf joints 431-436 has been placed into the floating state. As shown in FIG. 7, the specific joint of joints 431-436 that is placed into the floating state is joint 431, which is located at stationary base 402. In some embodiments, joint 431 is placed into the floating state while joints 432-436 remain in a locked state. Thus, in some embodiments, the most proximal joint 431 is placed into the floating state. As a result, links 421-426 and joints 432-436 rotate about joint 431 in response to pushing action 701. In some embodiments, some other joint of joints 431-436 is placed into the floating state, such as a joint 434 that is immediately proximal to single contact location 703. In such an embodiment, the operator interaction causes links 424-426 and joints 435 and 436 to rotate about joint 434 in response to pushing action 701. In some embodiments, a proximal joint that is an integer number “N” joints from single contact location 703 is placed into the floating state. For example, when N 3, joint 432, causing links 422-426 and joints 433-436 to rotate about joint 432 in response to pushing action 701. In some embodiments, two or more of joints 431-436 proximal to the operator interaction may be placed in the floating state. In some embodiments, proximal joints that are within N joints from single contact location 703 are placed into the floating state. For example, when N=3, joints 432, 433, and 434 are placed in the floating state.

FIG. 8 schematically illustrates kinematic chain 400 responding to an operator interaction with two contact locations 803a and 803b when no constraint is present, according to some embodiments. As shown, one or more operators interact with kinematic chain 400 via two pulling (or pushing) actions 801 against two different links of links 421-426: link 423 and link 424. In response, some of joints 431-436 are placed into a floating state, so that motion of such joints and one or more links 421-426 in contact with such joints is enabled. In the embodiment illustrated in FIG. 8, the pose of kinematic chain 400 is not currently constrained by a constraint.

In some embodiments, one or more of joints 431-436 proximal to the two contact locations 803a and 803b are placed into a floating state when actions 801 are detected and kinematic chain 400 is not currently constrained by a constraint. Thus, because the remaining joints of joints 431-436 remain in a locked state, end effector 412 and some or all of links 421-426 of kinematic chain 400 are moved by a continuing of actions 801 and/or a further operator interaction applied after the one or more of joints 431-436 have been placed into the floating state. As shown in FIG. 8, joint 431 is placed into the floating state, which is the most proximal joint of kinematic chain 400. In other embodiments, a joint of joints 431-436 that is immediately proximal to the most proximal contact location (e.g., contact location 803a) is placed into the floating state, such as joint 433. In some embodiments, the joint that is placed into the floating state is a proximal joint that is N joints from the most proximal contact location 803a. In some embodiments, one, two or more of joints 431-436 proximal to contact location 803a or 803b are placed into the floating state.

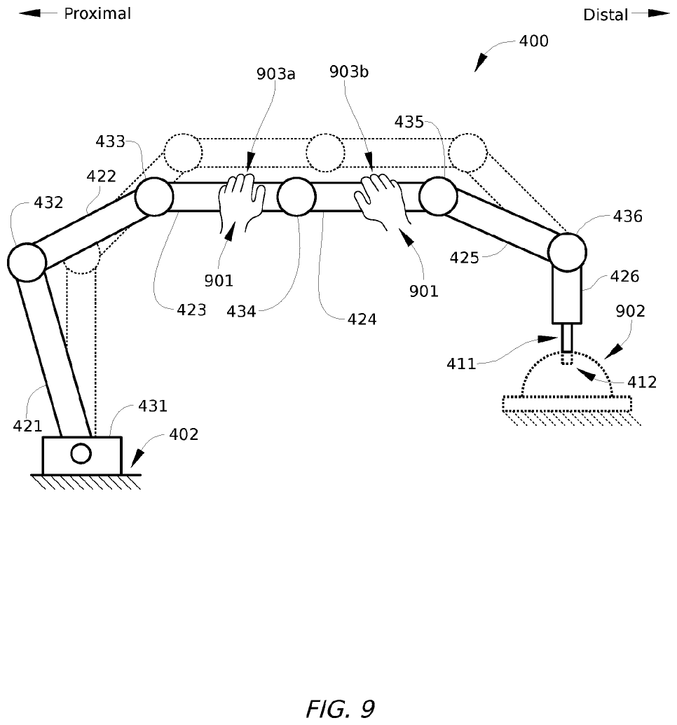

FIG. 9 schematically illustrates kinematic chain 400 responding to an operator interaction with two contact locations 903a and 903b when a constraint 902 is present, according to some embodiments. As shown, one or more operators interact with kinematic chain 400 via two pulling (or pushing) actions 901 against two different links of links 421-426: link 423 and link 424. In response, one or more of joints 431-436 are placed in a floating state, so that motion of such joints and of one or more of links 421-426 is enabled. In the embodiment illustrated in FIG. 9, the pose of kinematic chain 400 is currently constrained by constraint 902. In some embodiments, constraint 902 may be consistent with the constraint imposed on kinematic chain 400 in FIG. 4, for example the positioning of end effector 412 with respect to object 401.

In some embodiments, a joint of joints 431-436 disposed between contact locations 903a and 903b is maintained in a locked state, while one or more joints distal to the most distal contact location (contact location 903b) are placed into the floating state and/or one or more joints proximal to the most proximal contact location (contact location 903a) are placed into the floating state. In the embodiment illustrated in FIG. 9, joints 431, 432, 433, and 435 are placed into the floating state, joint 434 remains in a locked state, and joints 435 and/or 436 are selected to driven to perform compensating motion. Consequently, links 421-425 can be controlled to move as shown in response to pulling (or pushing) actions 901 and/or further pulling or pushing actions, while links 423 and 424 remain stationary with respect to each other and the pose of kinematic chain 400 adheres to constraint 902.

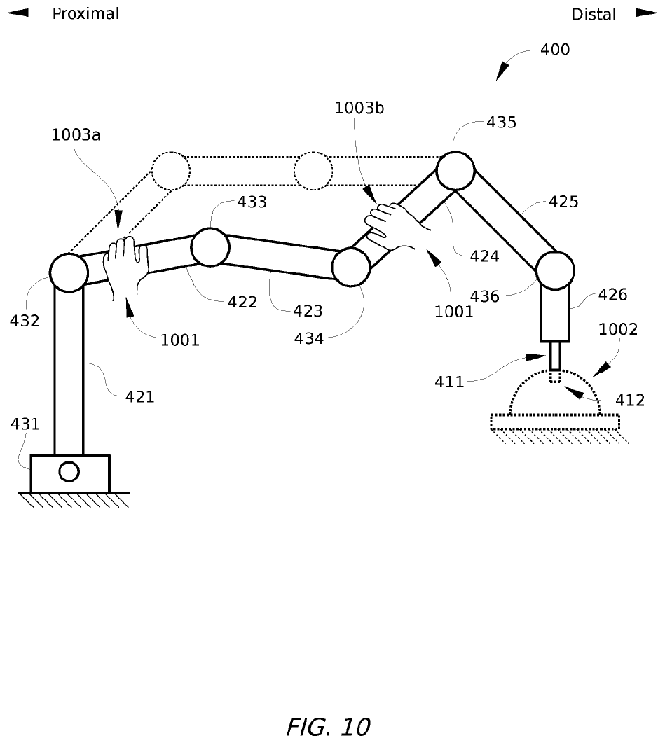

FIG. 10 schematically illustrates kinematic chain 400 responding to an operator interaction with two contact locations 1003a and 1003b when a constraint 1002 is present, according to some embodiments. As shown, one or more operators interact with kinematic chain 400 via two pulling (or pushing) actions 1001 against two different links of links 421-426: link 422 and link 424. Thus, one or more of joints 431-436 are disposed between contact location 1003a and contact location 1003b: joint 433 and joint 434. In the embodiment, when an operator interaction (pulling actions 1001 are shown, or a pushing action) is detected with contact at two contact locations while kinematic chain 400 is currently constrained by constraint 1002, one or more of joints 431-436 disposed between the two contact locations are placed into a floating state. Thus, motion of such joints and of at least links 422 and link 424 is enabled in response to operator interaction. As shown in FIG. 10, each of the joints 433 and 434 that are disposed between contact location 1003a and contact location 1003b are placed into the floating state. In some embodiments, additional joints of joints 431-436 that are not disposed between contact location 1003a and contact location 1003b are also placed into the floating state. In some examples, one or more of joints 431-436 that are proximal to the most proximal contact location (e.g., contact location 1003a) may also be placed in the floating state. In another example, one or more of joints 431-436 that are distal to the most distal contact location (e.g., contact location 1003b) may also be placed in the floating state.

Some examples of control units, such as control unit 130 may include non-transient, tangible, machine readable media that include executable code that when run by one or more processors (e.g., processor 140) may cause the one or more processors to perform the processes of method 1000. Some common forms of machine readable media that may include the processes of method 1000 are, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, and/or any other medium from which a processor or computer is adapted to read.

Although illustrative embodiments have been shown and described, it is to be understood that such embodiments are merely illustrative of and not restrictive on the broad inventive concept. A wide range of modification, change and substitution is contemplated in the foregoing disclosure and in some instances, some features of the embodiments may be employed without a corresponding use of other features. One of ordinary skill in the art would recognize many variations, alternatives, and modifications. Thus, the scope of the invention should be limited only by the following claims and equivalents thereof.

1. A computer-assisted device comprising:
a kinematic chain comprising a plurality of links coupled by a plurality of joints, the kinematic chain being configured to support an end effector; and
a control unit coupled to the kinematic chain;
wherein the control unit is configured to:
determine location information for a first operator interaction with the kinematic chain;
determine a current state of the computer-assisted device;
determine one or more joints to place into a floating state based on one or more parameters, the one or more parameters being of the first operator interaction or of computer-assisted device, the one or more parameters comprising the location information and the current state, the plurality of joints comprising the one or more joints; and
place the one or more joints into the floating state in response to determining the one or more joints.

2. (canceled)

3. The computer-assisted device of claim 1, wherein the control unit is further configured to, when the current state of the computer-assisted device is an error state, float no joints of the plurality of joints.

4-6. (canceled)

7. The computer-assisted device of claim 1 wherein:
the control unit is further configured to determine a type of interaction for the first operator interaction;
the one or more parameters further comprise the type of interaction; and
the one or more joints comprise different sets of the plurality of joints are determined as the one or more joints for different types of interaction.

8-10. (canceled)

11. The computer-assisted device of claim 1, wherein:
the location information includes an indication of one or more contact locations associated with the first operator interaction;
the control unit is further configured to determine a number of the one or more contact locations associated with the first operator interaction;
the one or more parameters further comprise the number of the one or more contact locations; and
different sets of the plurality of joints are determined as the one or more joints for different numbers of contact locations.

12-13. (canceled)

14. The computer-assisted device of claim 1, wherein the one or more parameters further comprise a constraint in one or more degrees of freedom of the kinematic chain.

15. The computer-assisted device of claim 14, wherein the constraint is imposed on the kinematic chain distally to a location of the first operator interaction.

16. The computer-assisted device of claim 14, wherein the constraint comprises:
an external obstacle to the kinematic chain; or
a range-of-motion limit for a joint of the plurality of joints.

17. (canceled)

18. The computer-assisted device of claim 14, wherein the constraint comprises a software constraint to maintain a position or an orientation of a portion of the kinematic chain located distally to a location of the first operator interaction.

19. (canceled)

20. The computer-assisted device of claim 14, wherein the constraint comprises a software constraint to maintain both a position and an orientation of a portion of the kinematic chain located distally to a location of the first operator interaction, and wherein the control unit is further configured to:
detect a motion of the one or more joints after placing the one or more joints in the floating state;
determine a compensating motion of at least one joint of the plurality of joints in response to the motion of the one or more joints, wherein the compensating motion of the at least one joint combined with the motion of the one or more joints maintain the constraint; and
actuate the at least one joint in accordance with the compensating motion.

21. (canceled)

22. The computer-assisted device of claim 1, wherein the control unit is further configured to:
determine a direction of an asserted force associated with the first operator interaction based on a signal received from a sensor system, wherein
the one or more parameters further comprise the direction of the asserted force.

23. The computer-assisted device of claim 22, wherein the one or more parameters further comprise that each joint of the one or more joints has, at a time of the first operator interaction, a degree of freedom in a direction that includes a component of the direction of asserted force.

24. The computer-assisted device of claim 1, wherein:
the location information indicates the first operator interaction is with a first link of the plurality of links; and
to determine the one or more joints to place into the floating state, the control unit is configured to select at least one joint of the plurality of joints, the at least one joint being located proximally to the first link along the kinematic chain or being located closest to and proximal to a location of the first operator interaction.

25-27. (canceled)

28. The computer-assisted device of claim 1, wherein the location information indicates a link of the plurality of links that is associated with the first operator interaction, and wherein the one or more parameters further comprise a joint type of a joint of the plurality of joints, the joint being located adjacent to the link.

29-30. (canceled)

31. The computer-assisted device of claim 1, wherein:
the control unit is further configured to determine second location information for a second operator interaction with the kinematic chain, and the one or more parameters further comprise the second location information; and
to determine the one or more joints of the plurality of joints to place into the floating state, the control unit is configured to select the one or more joints to comprise at least one joint of the plurality of joints located in the kinematic chain between a first location of the first operator interaction and a second location of the second operator interaction.

32. (canceled)

33. The computer-assisted device of claim 31, wherein to select the at least one joint, the control unit is configured to select a configurable number of joints adjacent to at least one of the first location and the second location.

34. The computer-assisted device of claim 31, wherein to select the at least one joint, the control unit is configured to select a first joint of the plurality of joints, the first joint being located distal to and adjacent to a more distal of the first and second locations.

35. The computer-assisted device of claim 1, wherein:
the control unit is further configured to determine second location information for a second operator interaction with the kinematic chain, and the one or more parameters further comprise the second location information; and
the control unit is configured to determine the one or more joints of the plurality of joints to place into the floating state by selecting the one or more joints to exclude at least one joint of the plurality of joints between a first location of the first operator interaction and a second location of the second operator interaction.

36. The computer-assisted device of claim 35, wherein at least one joint comprises a joint adjacent to a first link associated with the first location, or adjacent to a second link associated with the second location.

37. A method comprising:
determining, by a control unit of a computer-assisted device, location information for a first operator interaction with a kinematic chain of the computer-assisted device, the kinematic chain comprising a plurality of links coupled by a plurality of joints, the kinematic chain being configured to support an end effector;
determining, by the control unit, a current state of the computer-assisted device;
determining, by the control unit, one or more joints to place into a floating state based on one or more parameters, the one or more parameters being of the first operator interaction or of computer-assisted device, the one or more parameters comprising the location information and the current state of the computer-assisted device, the plurality of joints comprising the one or more joints; and
placing, by the control unit, the one or more joints into the floating state in response to determining the one or more joints.

38. (canceled)

39. The method of claim 37, wherein the method further comprises:
when the current state of the computer-assisted device is an error state, floating no joints of the plurality of joints.

40. (canceled)

41. The method of claim 37, further comprising:
determining, by the control unit, a type of interaction for the first operator interaction, wherein the one or more parameters further comprise the type of interaction, and wherein different sets of the plurality of joints are determined as the one or more joints for different types of interaction.

42-43. (canceled)

44. The method of claim 37, wherein:
the location information includes an indication of one or more contact locations associated with the first operator interaction; and
the method further comprises determining, by the control unit, a number of the one or more contact locations associated with the first operator interaction, wherein the one or more parameters further comprise the number of the one or more contact locations, and wherein different sets of the plurality of joints are determined as the one or more joints for different numbers of contact locations.

45. The method of claim 37, wherein the one or more parameters further comprise a constraint in one or more degrees of freedom of the kinematic chain.

46-47. (canceled)

48. The method of claim 37, further comprising:
determining, by the control unit, a direction of asserted force associated with the first operator interaction based on a signal received from a sensor system, wherein
the one or more parameters further comprise the direction of the asserted force.

49-53. (canceled)

54. The method of claim 37, wherein the location information indicates a link of the plurality of links that is associated with the first operator interaction, and wherein the one or more parameters further comprise a joint type of a joint of the plurality of joints, the joint being located adjacent to the link.

55. (canceled)

56. The method of claim 37, further comprising:
determining, by the control unit, second location information for a second operator interaction with the kinematic chain, and the one or more parameters further comprise the second location information, wherein determining the one or more joints of the plurality of joints to place into the floating state comprises selecting the one or more joints to comprise at least one joint of the plurality of joints located in the kinematic chain between a first location of the first operator interaction and a second location of the second operator interaction.

57. The method of claim 56, wherein selecting the at least one joint comprises selecting a first joint of the plurality of joints, the first joint being:
located proximal to and adjacent to a more proximal of the first and second locations; or located distal to and adjacent to a more distal of the first and second locations.

58. (canceled)

59. The method of claim 37, further comprising:
determining, by the control unit, second location information for a second operator interaction with the kinematic chain, and the one or more parameters further comprise the second location information; wherein
determining the one or more joints of the plurality of joints to place into the floating state comprises: selecting the one or more joints to exclude at least one joint of the plurality of joints between a first location of the first operator interaction and a second location of the second operator interaction.

60. A non-transitory machine-readable medium comprising a plurality of machine-readable instructions which when executed by one or more processors are adapted to cause the one or more processors to perform a method comprising:
determining location information for a first operator interaction with a kinematic chain of a computer-assisted device, the kinematic chain comprising a plurality of links coupled by a plurality of joints, the kinematic chain being configured to support an end effector;
determining a current state of the computer-assisted device;
determining one or more joints to place into a floating state based on one or more parameters, the one or more parameters being of the first operator interaction or of computer-assisted device, the one or more parameters comprising the location information and the current state, the plurality of joints comprising the one or more joints; and
placing the one or more joints into the floating state in response to determining the one or more joints.

61. The non-transitory machine-readable medium of claim 60, wherein the method further comprises:
determining a type of interaction for the first operator interaction; and wherein
the one or more parameters further comprise the type of interaction, wherein different sets of the plurality of joints are determined as the one or more joints for different types of interaction.

62. The non-transitory machine-readable medium of claim 60, wherein the location information indicates a link of the plurality of links that is associated with the first operator interaction, and wherein the one or more parameters further comprise a joint type of a joint of the plurality of joints, the joint being located adjacent to the link.

63. The non-transitory machine-readable medium of claim 60, wherein the method further comprises:
determining second location information for a second operator interaction with the kinematic chain, and the one or more parameters further comprise the second location information, wherein determining the one or more joints of the plurality of joints to place into the floating state comprises:
selecting the one or more joints to comprise at least a first joint of the plurality of joints located between a first location of the first operator interaction and a second location of the second operator interaction, or
determining the one or more joints to exclude the at least a second joint of the plurality of joints located between the first location and the second location.

64. The method of claim 45, wherein the constraint is imposed on the kinematic chain distally to the first operator interaction.
